SEQUENCE LISTING (2 sequences)

SEQ ID NO: 1 - DNA, 24 bp, Artificial Sequence
  Synthetically generated oligonucleotides
ggccatgaatttgacctctatgaa

SEQ ID NO: 2 - DNA, 24 bp, Artificial Sequence
  Synthetically generated oligonucleotides
ttcaacttctgaacactgaggaat

(19) United States
(12) Patent Application Publication
(10) Pub. No.: US 2009/0138976 A1
(43) Pub. Date: May 28, 2009

(54) ANIMAL MODEL FOR HEPATITIS C VIRUS INFECTION

(75) Inventors: Michael R. Charlton, Rochester, MN (US); Jeffrey L. Platt, Ann Arbor, MI (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 11/576,231

(22) PCT Filed: Sep. 29, 2005
(86) PCT No.: PCT/US05/35172

§ 371 Date: Sep. 9, 2008

Related U.S. Application Data
(60) Provisional application No. 60/614,281, filed on Sep. 29, 2004.

(51) Int. Cl. A01K 67/027

(52) U.S. Cl. 800/9; 800/18

(57) ABSTRACT

The current document relates to transgenic rodents that have a nucleic acid molecule encoding a hepatocyte mitogen polypeptide.

### BACKGROUND

1. Technical Field

This document relates to transgenic rodents having a nucleic acid molecule encoding an hepatocyte mitogen polypeptide.

2. Background Information

There are about three million individuals infected with the hepatitis C virus (HCV) in the United States, where HCV infection causes 20% of acute and 70% of chronic hepatitis (Alter, Hepatology, 1997, 26(3),62S-65S) and Murphy et al. (JAMA, 1996, 275: 995-1000). End-stage liver disease associated with HCV infection is the most common indication for liver transplantation in the United States. Using currently approved therapies, pegylated and standard interferons plus ribavirin, chronic HCV infection can be eradicated in about 50% of treated individuals.

To advance the study of HCV infection in humans and to test therapeutic approaches, a widely available and relatively inexpensive small animal model would be desirable. A rodent that is chimeric for human hepatocytes and that could support HCV infection in a sustainable manner would be useful. Thus, a mouse strain transgenic for human dHGF is featured herein. In addition, materials and methods are featured for achieving a prolonged human hepatocyte engraftment in a small animal model transgenic for dHGF.

### SUMMARY

This document provides materials and methods related to transgenic non-human animals (e.g., rodents) that have a nucleic acid molecule encoding an hepatocyte mitogen polypeptide. In general, this document provides transgenic rodents having a nucleic acid molecule encoding an hepatocyte mitogen polypeptide. Such rodents can contain human liver cells and can maintain a hepatitis C virus infection. Non-human animals having human liver cells and maintaining a hepatitis C virus infection can be used to assess the initiation, progression, and treatment of an active hepatitis C virus infection within an in vivo model. In some cases, the non-human animals can be immunocompetent, allowing for the analysis of how hepatitis C virus interacts in vivo with the infected animal's immune system. Such non-human animals also can be used to assess potential hepatitis C virus treatment compounds in vivo.

In general, one aspect of this document provides a transgenic rodent containing a nucleic acid molecule encoding an hepatocyte mitogen polypeptide, wherein the rodent contains human liver cells. The rodent can be immunocompetent or immunocompromised. The rodent can be a SCID mouse, a nude mouse, or a Rag 1 and/or Rag 2 deficient mouse. The rodent can contain hepatitis C virus. The hepatocyte mitogen polypeptide can be a hepatocyte growth factor polypeptide. The hepatocyte mitogen polypeptide can be a human hepatocyte growth factor polypeptide. The hepatocyte mitogen polypeptide can be a human hepatocyte growth factor polypeptide lacking five amino acid residues. The hepatocyte mitogen polypeptide can be dHGF. The nucleic acid can contain an albumin promoter or enhancer sequence. The human liver cells can be hepatocytes. The rodent can be capable of maintaining the human liver cells for at least one month. The rodent can be capable of maintaining the human liver cells for at least two months. The human liver cells can be primary cells obtained from a human liver sample. The human liver cells can be infected with hepatitis C virus. The rodent can be capable of maintaining human liver cells infected with hepatitis C virus for at least one month. The rodent can be capable of maintaining human liver cells infected with hepatitis C virus for at least 3 months.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a diagram of a construct for making a transgenic mouse that expresses human dHGF.

FIG. 2 contains a Western blot analysis of human albumin immunoreactivity in representative transgenic (top panel) and non-transgenic (middle panel) animals as well as human albumin immunoreactivity in six transgenic animals at day 78 post-transplantation (bottom panel). MWS: Molecular Weight Standard of 50 kDa marker shown; Albumin: Positive control; FVB/Cr: Serum of non-transgenic, non-transplanted FVB/Cr mouse (negative control); Human serum: Human serum (positive control). Day number refers to the number of days following transplantation of human hepatocytes until the sample was collected from a study animal. The letters A-F in the Bottom Panel refer to the identity of the study animal. Animals A-F were transgenic for dHGF.

### DETAILED DESCRIPTION

This document provides materials and methods related to transgenic non-human animals (e.g., rodents) that have a nucleic acid molecule encoding an hepatocyte mitogen polypeptide (e.g., human dHGF). Such transgenic non-human animals can contain and maintain a prolonged human hepatocyte engraftment, providing, for example, a small animal model transgenic for hepatitis C infection.

Any type of non-human animal can be designed to contain a nucleic acid molecule encoding an hepatocyte mitogen polypeptide. For example, standard transgenic technology can be used to make transgenic mice, rats, rabbits, guinea pigs, or hamsters.

Nucleic acid encoding any type of hepatocyte mitogen polypeptide can be used to make a transgenic non-human animal containing a nucleic acid molecule encoding an hepatocyte mitogen polypeptide. Hepatocyte mitogens include, without limitation, complete mitogens that are capable of stimulating DNA synthesis in hepatocytes without co-factors. Complete mitogens can be epidermal growth factor (EGF) (De Smet et al., Bioch. Pharm., 61: 1293-1303 (2001); Moriuchi et al., Bioch. Biophy. Res. Comm., 280: 368-373 (2001); Zheng et al., Transp. Proc., 32:2498-2499 (2000)), transforming growth factor alpha (TGF-α) (Webber et al., Hepatology, 19:489-497 (1994)), tumor necrosis factor alpha (TNF-α) (Iocca et al., Am. J Path., 163: 465-476 (2003); Yamada et al., Hepatology, 28:959-970 (1998); Webber et al., Hepatology, 28:1226-1234 (1998)), and hepatocyte growth factor (HGF) (Nakamura et al., Nature, 342:440-443 (1989) and Nakamura, Progress in Growth Factor Research, 3:67-85 (1991)). Interleukin-6 (IL-6), produced by Kupffer cells, can be used as a component in induction of hepatocyte replication (Cressman et al., Science, 274:1379-1383 (1996)). TNF-α is thought to regulate secretion of IL-6, a signal for acute phase protein synthesis by hepatocytes. HGF is a very potent complete hepatocyte mitogen. This polypeptide is 100 fold more active than TGF-α and EGF. HGF and its receptor, c-Met, play a role in liver growth and hepatocyte replication. The role of HGF is revealed by homozygous deletions of HGF, which cause embryonic death associated with hepatic agenesis (Strain et al., J. Clin. Inves., 87:1853-1857 (1991); Naldini et al., Oncogene, 6:501-504 (1991); Ponzetto et al., Cell, 77: 261-271 (1994); and Schmidt et al., Nature, 373:699-702 (1995)).

Since the initial cloning of the human HGF cDNA (Nakamura et al., Proc. Natl. Acad. Sci., 83:6489-6493 (1986); and Gohda et al., J Clin. Inves., 81:414-419 (1988)), HGF has been shown to be encoded by a single open reading frame (Nakamura et al., Nature, 342: 440-443 (1989); and Miyazawa et al., Bioch. Biophy. Res. Comm, 163:967-973 (1989)). In vivo, HGF exists in a 728 amino acid isoform and a 723 amino acid isoform (Itoh et al., Digest. Diseas. Sci., 27:341-346 (1982) and Bell et al., Oncogene, 18:887-895 (1999)). The 723 amino acid isoform is generated through alternative splicing of the primary RNA transcript, and it contains an in-frame deletion of five amino acids in the first kringle domain (amino acids 163-167). The smaller, 723 amino acid isomer of HGF is referred to as dHGF and is a more potent mitogen than the 728 amino acid isoform (Shima et al., Bioch. Biophy. Res. Comm, 200:808-815 (1994)). Mice transgenic for dHGF have an enhanced rate of regeneration of hepatocyte mass following partial hepatectomy, when compared to non-transgenic animals.

The transgenic non-human animals provided herein can be immunocompetent or immunocompromised. For example, any immunocompetent mouse strain can be used to make an immunocompetent transgenic mouse having a nucleic acid molecule encoding human dHGF. Examples of immunocompromised animals that can be used to make immunocompromised transgenic animals having a nucleic acid molecule encoding human dHGF include, without limitation, severe combined immune deficient (SCID or scid/scid) mice, nude mice (the nu/nu mouse is born without a thymus), and animals containing a genetic defect in, for example, a Rag sequence (e.g., a Rag 1 and/or Rag 2 deficient mouse which lacks mature T and B lymphocytes). In addition, normally immunocompetent animals can be transformed into immunocompromised animals by, for example, administration of an immunosuppressant (e.g., cyclosporin) or removal of the thymus.

The transgenic non-human animals provided herein can contain any type of human cell. For example, transgenic mice containing a nucleic acid molecule encoding human dHGF can contain human liver cells. Such mice can maintain human liver cells from one, two, three, four, five, six, or more months. Any method can be used to obtain human cells. For example, human liver cells can be obtained from previously maintained liver cell cultures or from a liver biopsy sample. Any method can be used to deliver human cells to a transgenic non-human animal. For example, standard tissue transplant techniques can be used to surgically place human liver cells into a transgenic non-human animal. The human cells within a transgenic non-human animal provided herein can be infected with hepatitis C virus. The human cells can be infected ex vivo. For example, a transgenic mouse can be given human liver cells that are infected with hepatitis C virus before being transplanted within the transgenic mouse. In some cases, the human cells can be infected in vivo. For example, a transgenic mouse can be given human liver cells that are infected with hepatitis C virus after being transplanted within the transgenic mouse.

The invention will be further described in the following examples, which do not limit the scope of the invention described in the claims.

### EXAMPLES

### Example 1

### Chimeric Mouse Transgenic for a Five Amino Acid Deletion Isoform of Human Hepatocyte Growth Factor

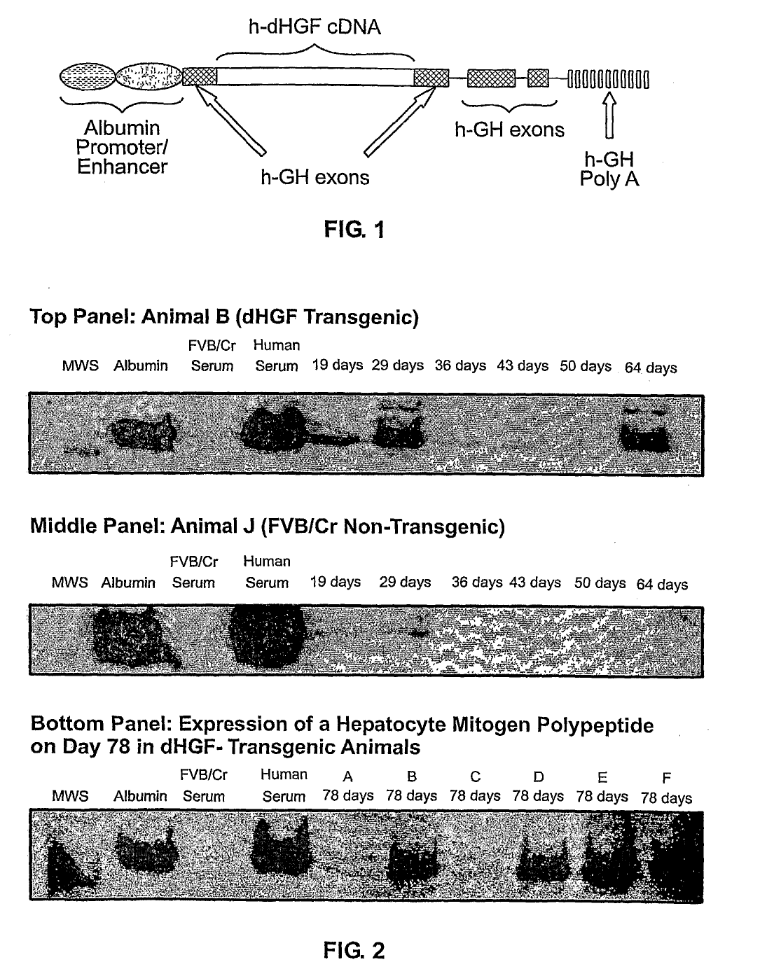

A 7.5 kb construct containing a 2.3 kb coding region of the 723 amino acid isoform HGF cDNA under the transcriptional control of a mouse albumin promoter enhancer was used to generate transgenic mice with a FVB/Cr mouse background (Charles River Labs, Wilmington, Mass.) by established methods (Hogan B., Manipulating the Mouse Embryo: A Laboratory Manual. 1 ed. New York: Cold Spring Harbor laboratory, 1986). The 723 isoform of human HGF (dHGF) lacks a five amino acid sequence (Phe, Leu, Pro, Ser, Ser) when compared to the full-length cDNA. The 2.3 kb dHGF cDNA (black box; FIG. 1) was inserted into the first exon of the human growth hormone gene (gray boxes; FIG. 1) and controlled by the murine albumin promoter/enhancer (ovals; FIG. 1). A human growth hormone polyadenylation site was attached (FIG. 1).

The dHGF construct was cut from the pSPORT1 plasmid with the restriction enzyme BamHI (Roche Molecular Biochemicals, Indianapolis, Ind.). The digestion reaction was separated on a 1% agarose gel, (SeaPlaque GTG Agarose, Cambrex Bio Science, Rockland, Me.). The dHGF band was excised, and DNA purification was performed (Qiaquick Gel Extraction Kit, Qiagen Corporation, Valencia, Calif.). The pcDNA 5/TO was rehydrated to a concentration of 0.5 μg/μL. The vector was then digested with BamHI, and the dHGF was ligated at this site using T4 DNA Ligase and T4 DNA Ligase buffer from Invitrogen (Invitrogen Corporation, Carlsbad, Calif.). Transformation was accomplished with the use of One Shot Top 10 chemically competent cells from Invitrogen (Invitrogen Corporation, Carlsbad, Calif.). Plating of the transformed cells was done on LB-Agar (Qbiogene, Incorporated, Carlsbad, Calif.), and colonies were picked the next day for overnight growth (at 37° C.) in LB-Media (Qbiogene, Carlsbad, Calif.) with the addition of 5 mg of Ampicillin (Sigma-Aldrich, St. Louis, Mo.). Plasmid DNA preparation was accomplished by using the Qiafilter Plasmid Midi kit (Qiagen). The clone DNA was then digested with BamHI (Roche Molecular Biochemicals, Indianapolis, Ind.) and separated by gel electrophoresis on a 1% agarose (Cambrex Crop., East Rutherford, N.J.) gel to determine insert incorporation.

Several of the pcDNA5/dHGF clones that contained the insert were sequenced. Insert orientation was determined based upon the sequencing results. For microinjection, pcDNA5/dHGF was cut at the NruI (upstream of the CMV promoter) and SmaI sites (downstream of SV40 intron and Poly A) to remove vector sequences. The fragment was then separated on a 1% agarose gel, and the band was excised and purified using the Qiaquick Gel Extraction kit from Qiagen (Qiagen Corporation, Valencia, Calif.). The fragment was purified by using a 0.22 micron spin filter, and finally brought to a final concentration of 3 ng/mL with injection buffer (10 mM Tris/0.1 mM EDTA/pH 8.0).

Transgenic mice were identified by Southern blot analysis of EcoRI digested genomic tail DNA using a ³²P-labelled 2.3 kb dHGF cDNA probe as described elsewhere (Liu et al., 1994, Gene, 144: 179-187). PCR of blood DNA was also performed to confirm transgenicity using primers specific for the dHGF gene (Chen et al., 1997, Hepatology, 26: 59-66). Founder (F₀) mice were propagated with FVB/Cr mice to homozygosity for dHGF.

dHGF Expression

Expression of dHGF mRNA was confirmed by reverse transcription PCR (RT-PCR) with tissue of animals found to be positive by PCR for the dHGF transgene as described elsewhere (Bell et al., 1999, Oncogene, 18: 887-895). Briefly, 1 mg of total RNA was reverse-transcribed using avian myeloblastosis virus reverse transcriptase (Roche Diagnostics) and amplified with Taq DNA polymerase using primers specific for HGF/dHGF (sense, 5′-GGCCATGAATTTGACCTCTATGAA-3′, SEQ ID NO:1; and antisense, 5′-TTCAACTTCTGAACACTGAGGAAT-3′, SEQ ID NO:2). The primers used were oriented on either side of the 15 bp deletion of dHGF and produce a 278 bp product for dHGF and a 293 bp product for fall length HGF. These primers have weak cross-species hybridization for the fall length HGF gene.

The lower band (278 bp product) was excised from the agarose gel, and DNA was purified using a DNA gel extraction spin column (Biorad Laboratories, Hercules, Calif.). Purified DNA was sequenced. DNA sequence from the transgenic mouse had complete homology with the HGF sequence (GenBank Accession No. E15445) and included a 15 bp deletion in the first kringle domain. DNA sequence from the non-transgenic mice had complete homology with mouse HGF sequence (GenBank Accession No. XM_{—}131908 or NM_{—}010427).

To determine the relative expression of the dHGF transgene in different tissues, RT-PCR was carried out in the same fashion in heart, kidney, liver, lung, and spleen of dHGF transgenic mice. mRNA for the dHGF isoform was greatest in the liver.

### Example 2

### Transgenic dHGF Mice Containing Human Liver Cells

Once a colony of dHGF animals expressing the transgene was established, experiments were designed to determine whether dHGF transgenic mice were capable of accepting xenografts of human hepatocytes.

### Harvesting and Transplanting Human Hepatocytes

Cryopreserved human hepatocytes from whole livers deemed unsuitable for human transplantation (due to >30% steatosis) were used for hepatocyte harvest. Briefly, donor livers were determined to be free from infection from HBV and HCV infection, as measured by HBV sAg, cAb, and qualitative PCR for HCV RNA. Donor livers were kept on ice in UW solution (Bair Laboratories, Pomona, N.Y.) until hepatocyte harvest. Hepatocytes were harvested using a three-step collagenase perfusion method as described elsewhere (Sielaff et al., Transplantation, 1995, 59: 1459-1463). A branch of the portal vein was cannulated prior to perfusion. The donor liver was placed in a sterile basin, submerged in pre-warmed saline (37° C.) and flushed with 5.0 L warm saline at 1.5 L/minute for 3-4 minutes to rapidly bring up the core temperature to 37° C. The liver was then perfused with 10 L of calcium free perfusate containing 2.5 mM ethylene glycol-bis (β-aminoethyl ether)-N-N-tetra acetic acid (EGTA, Sigma, St. Louis, Mo.) at 1.5 L/minute through an oxygenator (Unisyn Technologies, San Diego, Calif.). All solutions were pre-warmed to 37° C. prior to use. Thereafter, the liver tissue was perfused continuously with a solution of 0.1 % of collagenase D (Roche Diagnostics Corporation, Indianapolis, Ind.) and recirculated for 30 minutes until the liver was soft and well digested as determined by palpation. The liver was then immersed in cold William's E media (Sigma, St. Louis, Mo.) supplemented with 10% FBS, penicillin, and streptomycin. The liver capsule was incised, and the hepatocytes were gently combed out into solution. The hepatocyte suspension was then filtered through a Buchner funnel to remove any tissue fragments and then filtered through a 112 mm nylon mesh. Cell suspensions were washed three times in cold William's E medium, centrifuging at 50 g for 5 minutes between washes. Hepatocyte viability was assessed by hemocytometer and Trypan blue exclusion. Hepatocyte viability of greater than 88 percent was routinely achieved.

Harvested hepatocytes were suspended in Williams E medium containing FBS (10% v/v), dimethyl sulfoxide (10% v/v) and antibiotics (penicillin and streptomycin) at a cell concentration of 10⁷ cells/mL and cryopreserved in 1 mL cryovials and 25 mL bags. Cells were frozen stepwise using a computer controlled freezing container (Custom BioGenic Systems, Shelby Township, Mich.). The applied freezing cycles were −1.0° C./min from 20 to −6° C., −25° C./min from −6 to −50° C., −15° C./min from −50° C. to -14° C., −1° C./min from −14° C. to −45° C., and −10° C./min from −45 to −90° C. Cryopreserved hepatocytes were stored at −196° C. in liquid nitrogen.

Prior to transplantation, vials containing hepatocytes were removed from liquid nitrogen and rapidly thawed to 37° C. in a water bath. Hepatocytes were washed twice successively in William's E medium and centrifuged at 50×g for 5 minutes at 4° C. Washed hepatocytes were re-suspended in sterile PBS at 50×10⁶/mL. 1×10⁶ human hepatocytes were resuspended in a volume of 20 mL and used for transplantation into recipient mice.

As a preparation to the hepatocyte transplantation, mice were anesthesized with Ketamine and Xylazine, and their left flank was shaved and scrubbed. An incision of ˜0.5 cm was then made caudal to the left false ribs, and the inferior end of the spleen was exteriorized. 1×10⁶ cryopreserved human hepatocytes were suspended in total volume of 0.02 mL sterile PBS, and injected into the inferior end of the spleen using a 25 gauge needle. Following the injection, the spleen was returned to its physiological position, and the peritoneum was closed with a continuous pattern of 8-0 absorbable Vicryl. Next, the skin was closed with a continuous pattern of 6-0 absorbable Vicryl. The mice then were given buprenorphine (Reckitt Benckiser, Richmond, Va.) as described elsewhere (Deng et al, Comparative Medicine, 50: 628-632 (2000)) protocol, placed on a warm pad (37° C.) until they recovered from the anesthesia, and then returned to their cage. Following the surgery, the mice were monitored closely until they returned to their normal locomotion and feeding capabilities.

Hepatocytes were injected intrasplenically into dHGF transgenic (n=10) and non-transgenic FVB/Cr (n=10) mice when between 6-7 days old (˜2-3 g in weight).

Serum samples were collected at frequent time points from all mice until 12 weeks post-transplantation. At the completion of the studies, liver tissue was collected for immunohistochemistry analysis.

### Detection of Human Serum Albumin by Immunoblotting

Samples of mouse serum were subjected to SDS-PAGE and blotted onto PVDF membranes. The blots were probed with a commercially available mouse monoclonal antibody against human serum albumin (ZMHSA1.1, 1:10,000 dilution; Pierce, Rockford, Ill.) that was biotinylated (Molecular Probes Inc., Eugene, Oreg.) and which does not cross react with mouse proteins (Mercer et al., Nature Medicine, 2001, 7: 927-933). Specific binding was detected using horseradish-labeled streptavidin (1:5,000; KPL, Gaithersburg, Md.) and an enhanced chemiluminescence system (Pierce, Rockford, Ill.). All serum samples were routinely diluted 1:100 prior to loading on gel for electrophoresis.

The relative abundance of human albumin in experimental groups/protocols was measured by comparing band intensity as calculated by Kodak Image Station (Kodak, Rochester, N.Y.). Band intensity was quantified as a ratio of band intensity to control. Samples producing strongly positive bands for which relative abundance of human albumin cannot be resolved by Image Station analysis underwent further serial dilution. Band intensity ratios were summarized using means, medians, and percentiles. Groups were compared using chi-square and Wilcoxon Rank Sum tests.

The immunoblotting results demonstrate that human hepatocyte engraftment, as measured by human albumin production, was achievable in mice transgenic for the dHGF gene (FIG. 2). The longitudinal pattern of human albumin production in the seven (out of ten) transgenic animals that were strongly positive for human albumin at 78 days post-transplantation was of particular interest. In each dHGF transgenic animal, human albumin was initially detectable from the time of transplantation to day 29. After day 29, the abundance of human albumin declined (although never to undetectable levels), suggesting either decreased human hepatocyte protein synthesis and/or decreased human hepatocyte mass. The subsequent rise in human albumin production, seen on and after day 64, was most likely to have been on the basis of increased human hepatocyte mass. The initial decline and subsequent rise in human albumin levels and, probably, human hepatocyte mass, suggests the selection and expansion of clones of human hepatocyte more responsive to dHGF or less susceptible to immunological clearance in these animals. The basis of this apparent clonal selection and expansion of human hepatocytes might, for instance, reflect relative expression of c-Met, the receptor for dHGF, or post-receptor sensitivity to dHGF signaling.

The basis of the failure of human hepatocyte engraftment in the three dHGF transgenic mice that lost detectable levels of human albumin cannot be determined from these experiments. Although zygosity of the study animals was similar, as determined by Southern blot analysis, the site of integration, and thus expression, of the dHGF transgene for individual animals may have varied.

### Immunohistochemistry—Detection of Human Hepatocytes

Human hepatocyte engraftment was confirmed through immunohistochemical staining of study animal livers. The antibody used for human hepatocyte staining was the same as that described elsewhere (Mercer et al., Nature Medicine, 2001, 7: 927-933).

Mouse liver biopsies were fixed in 10% formalin and then embedded in paraffin. Sections (5 μm) were pre-treated with an avidin/biotin blocking kit (Zymed Laboratories, San Francisco, Calif.) and then immunostained with a monoclonal antibody specific for human hepatocytes (Clone OCH1E5, 1:20 dilution; DAKO, Carpinteria, Calif.). Bound antibody was detected by super sensitive immunodetection system (Biogenex, San Francisco, Calif.). Liver tissue from non-transgenic FVB/Cr, non-transplanted mice and dHGF mice that were not transplanted were also stained.

Immunohistochemical liver histology of study animals revealed that a normal murine hepatic architecture and hepatocyte morphology. Human hepatocyte immunostaining was negative in liver from a non-transgenic FVB/Cr mouse 12 weeks after transplantation with human hepatocytes. Extensive staining of human hepatocytes was detected with islands of non-staining murine hepatocytes in liver from a dHGF transgenic mouse 12 weeks after human hepatocyte transplantation. Human hepatocyte immunostaining was negative in liver from a dHGF transgenic mouse that has not undergone human hepatocyte transplantation.

These results demonstrate substantial engraftment of human hepatocytes. Chimerism was apparent with areas of intensely immunostaining hepatocytes adjacent to clusters of non-staining, presumably murine, hepatocytes. Portal areas were relatively devoid of staining for human hepatocytes. In contrast to similar studies described in Alb/u-PA mice following human hepatocyte transplantation, the gross appearance of the livers evaluated herein was normal (vs. nodular in the Alb/u-PA mouse). Transgenic Alb/u-PA mice characteristically undergo continuous hepatocellular injury and regeneration, with gross nodularity of the liver. In contrast, livers from dHGF transgenic mice are normal in appearance (although a minority of older mice may subsequently develop hepatocellular carcinoma).

### Example 3

### Transgenic dHGF Mice Containing Human Liver Cells Maintain an HCV Infection

All ten dHGF transgenic mice that received human hepatocyte transplantation were inoculated with human sera from patients with chronic HCV infection and high levels of HCV RNA. Briefly, mice were injected intravenously (internal jugular) 4 weeks after human hepatocyte transplantation with 100 ml of infectious serum obtained from a human chronically infected with HCV genotype 1.

Three of seven mice showing evidence of human hepatocyte engraftment, as measured by strongly positive immunoblotting for human albumin, developed low level increases in HCV RNA in serum. Peak HCV RNA levels were 2.84-3.14 log10 IU/mL. HCV RNA quantitation was carried out on aliquots of 100 mL of sera following extraction by a guanidine thiocyanate lysis protocol using reagents supplied in the Amplicor HCV Test and the Amplicor HCV Monitor Test kits (Roche Diagnostics, Branchburg, N.J.). The sensitivity Amplicor HCV Test and the Amplicor HCV Monitor Test kits are 50 IU/ml and 600 IU/ml respectively, with over 95% positivity rates. The Amplicor HCV Monitor Test assay has good linearity across genotypes and HCV RNA concentrations. HCV-RNA quantitation was carried out in a blinded fashion. None of the remaining mice, including the three with negative studies for human albumin, had detectable levels of HCV RNA at any time-point post-inoculation.

The combination of persistent and increasing production of human albumin and positive immunostaining demonstrated sustained engraftment of human hepatocytes in mice transgenic for dHGF. This sustained engraftment of human hepatocytes was in non-immunocompromised mice. The finding of early evidence of HCV infection in a proportion of chimeric dHGF transgenic mice, as measured by HCV positive strand RNA titers, also demonstrates the potential for the dHGF transgenic mice to be used as a small animal model of HCV infection. The dHGF transgenic model can be back-bred with SCID mice and/or administered retrorsine to enhance human hepatocyte engraftment.

In summary, the results provided herein demonstrate that human hepatocytes transplanted into dHGF transgenic mice persist and increase in biological activity, as measured by human albumin production. In addition, a proportion of dHGF transgenic mice that are chimeric for human hepatocytes are able to support low levels of HCV infection and possibly higher levels of HCV infection.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A transgenic rodent comprising a nucleic acid molecule encoding a hepatocyte mitogen polypeptide, wherein said rodent comprises human liver cells.

2. The transgenic rodent of claim 1, wherein said rodent is immunocompetent.

3. The transgenic rodent of claim 1, wherein said rodent is immunocompromised.

4. The transgenic rodent of claim 1, wherein said rodent is a SCID mouse.

5. The transgenic rodent of claim 1, wherein said rodent is a nude mouse.

6. The transgenic rodent of claim 1, wherein said rodent is a Rag 1 or Rag 2 deficient mouse.

7. The transgenic rodent of claim 1, wherein said rodent comprises hepatitis C virus.

8. The transgenic rodent of claim 1, wherein said hepatocyte mitogen polypeptide is a hepatocyte growth factor polypeptide.

9. The transgenic rodent of claim 1, wherein said hepatocyte mitogen polypeptide is a human hepatocyte growth factor polypeptide.

10. The transgenic rodent of claim 1, wherein said hepatocyte mitogen polypeptide is a human hepatocyte growth factor polypeptide lacking five amino acid residues.

11. The transgenic rodent of claim 1, wherein said hepatocyte mitogen polypeptide is dHGF.

12. The transgenic rodent of claim 1, wherein said nucleic acid comprises an albumin promoter.

13. The transgenic rodent of claim 1, wherein said nucleic acid comprises an albumin enhancer sequence.

14. The transgenic rodent of claim 1, wherein said human liver cells are hepatocytes.

15. The transgenic rodent of claim 1, wherein said rodent is capable of maintaining said human liver cells for at least one month.

16. The transgenic rodent of claim 1, wherein said rodent is capable of maintaining said human liver cells for at least two months.

17. The transgenic rodent of claim 1, wherein said human liver cells are primary cells obtained from a human liver sample.

18. The transgenic rodent of claim 1, wherein said human liver cells are infected with hepatitis C virus.

19. The transgenic rodent of claim 1, wherein said rodent is capable of maintaining human liver cells infected with hepatitis C virus for at least one month.

20. The transgenic rodent of claim 1, wherein said rodent is capable of maintaining human liver cells infected with hepatitis C virus for at least three months.

21. A transgenic mouse comprising a nucleic acid molecule encoding human dHGF, wherein said nucleic acid comprises an albumin promoter sequence, wherein said mouse is not immunocompromised, wherein said mouse comprises human hepatocytes and is capable of maintaining said human hepatocytes for at least two months, wherein said mouse comprises hepatitis C virus and is capable of maintaining human hepatocytes infected with said hepatitis C virus for at least two months.
